(19) United States
(12) Patent Application Publication
(10) Pub. No.: US 2022/0142556 A1
(43) Pub. Date: May 12, 2022

(54) SYSTEMS AND METHODS FOR ESTIMATING A NERVOUS SYSTEM STATE BASED ON MEASUREMENT OF A PHYSIOLOGICAL CONDITION

(71) Applicant: University of Houston System, Houston, TX (US)

(72) Inventors: Rose T. Faghih, Houston, TX (US); Dilranjan S. Wickramasuriya, Houston, TX (US); Md. Rafiul Amin, Houston, TX (US)

(21) Appl. No.: 17/514,129

(22) Filed: Oct. 29, 2021

Related U.S. Application Data
(60) Provisional application No. 63/110,480, filed on Nov. 6, 2020.

(51) Int. Cl. A61B 5/00; A61B 5/0531; A61B 5/145; A61B 5/0205; A61B 5/024; G16H 50/30; G16H 40/67

(52) U.S. Cl. CPC A61B 5/4035; A61B 5/0531; A61B 5/14546; A61B 5/7267; G16H 40/67; A61B 5/024; A61B 5/4836; A61B 5/4839; G16H 50/30; A61B 5/0205

(57) ABSTRACT

A system for estimating a state of the nervous system includes at least one sensor configured to sense a continuously variable non-neural physiological condition as sensed data, a relatively low performance processing device configured to receive the sensed data and estimate a state of a nervous system based on the sensed data, and a relatively high performance computing device configured to provide updates to the processing device to improve the estimate of the state of the nervous system. A method for estimating a state of the nervous system includes obtaining sensed data indicative of a continuously variable non-neural physiological condition, estimating a state of a nervous system based on the sensed data, outputting the estimated state of the nervous system, and receiving updates to improve the estimating.

### CROSS-REFERENCE TO RELATED APPLICATION

This application claims the benefit of and priority to U.S. Provisional Patent Application Ser. No. 63/110,480, filed on Nov. 6, 2020, the entire contents of which are incorporated by reference herein.

### STATEMENT REGARDING FEDERALLY SPONSORED RESEARCH/DEVELOPMENT

This invention was made with Government support under grant numbers 1942585 and 1755780 awarded by the NSF. The Government has certain rights in the invention.

### FIELD

The present disclosure relates to systems and methods for estimating a nervous system state, e.g., estimating a state of sympathetic arousal and/or estimating cortisol-related energy production, based on measurement of a (non-neural) physiological condition, e.g., skin conductance and/or cortisol levels.

### BACKGROUND

When physiological systems of the human body deviate from their usual range of functioning, medical disorders may result and/or other adverse effects may be triggered. Yet, the states of some of these physiological systems remain unobservable or difficult to observe. In particular, determining what is happening inside a person's brain or a state of the nervous system remains a challenge.

Wearable technology enables monitoring of various different physiological conditions and, for this reason, is likely to play a crucial role in the future of healthcare. However, currently available wearable technology is unable to provide deeper information or insight regarding states of physiological systems beyond the physiological condition information itself. That is, wearable technology is presently incapable of determining what is happening inside a person's brain or a state of the nervous system.

### SUMMARY

Terms including “generally,” “about,” “substantially,” and the like, as utilized herein, are meant to encompass variations, e.g., tolerances, measurement variations, and/or other variations, up to and including plus or minus 10 percent. Further, any or all of the aspects described herein, to the extent consistent, may be used in conjunction with any or all of the other aspects described herein.

Provided in accordance with aspects of the present disclosure is a system for estimating a state of the nervous system including at least one sensor, a relatively low performance processing device, and a relatively high performance computing device. The at least one sensor is configured to sense a continuously variable non-neural physiological condition and to output sensed data indicative of the continuously variable non-neural physiological condition. The relatively low performance processing device is operably coupled to the at least one sensor and configured to receive the sensed data therefrom. The processing device includes a processor and memory storing a first algorithm that, when executed by the processor, causes the processor to run the first algorithm to estimate a state of a nervous system based on the sensed data. The processing device is configured to output the estimated state of the nervous system. The relatively high performance computing device is operably coupled to the processing device and includes a processor and memory storing a second algorithm that, when executed by the processor, causes the processor to run the second algorithm to determine updates. The computing device is configured to communicate the updates to the processing device to improve the estimate of the state of the nervous system.

In an aspect of the present disclosure, the at least one sensor includes at least one skin conductance sensor and the continuously variable non-neural physiological condition is skin conductance. In such aspects, the estimate of the state of the nervous system may be at least one of: an identification of an autonomic nervous system activation or an estimate of a state of sympathetic arousal.

In another aspect of the present disclosure, the at least one sensor includes at least one blood cortisol sensor and the continuously variable non-neural physiological condition is a user's blood cortisol level. In such aspects, the estimate of the state of the nervous system may be an estimate of a state of cortisol-related energy production.

In another aspect of the present disclosure, the processing device is further configured to receive an external input and the processor is caused to estimate the state of the nervous system based on the sensed data and the external input.

In still another aspect of the present disclosure, the first algorithm is executed to estimate the state of the nervous system based on the sensed data in real-time, and the second algorithm is executed to determine updates occasionally.

In yet another aspect of the present disclosure, both the first and second algorithms perform estimation and the second algorithm is utilized to provide updated parameters to the first algorithm for estimating the state of the nervous system based on the sensed data using the first algorithm.

In still yet another aspect of the present disclosure, the first algorithm includes a forward filter algorithm and the second algorithm includes the forward filter algorithm and a backward smoothing algorithm. In such aspects, the computing device may be configured to repeatedly run the forward filter and backward smoothing algorithms to obtain updated model parameters, and to include the updated model parameters in the updates provided from the computing device to the processing device. Alternatively, in aspects, the updated model parameters may be determined by repeatedly running an expectation-maximization algorithm.

In another aspect of the present disclosure, the first algorithm includes a sparse recovery algorithm and the second algorithm includes a sparse recovery algorithm and a further estimation algorithm. The further estimation algorithm may include an expectation maximization algorithm or a coordinate descent algorithm. Additionally or alternatively, the sparse recovery algorithm may include a least squares algorithm or a Bayesian filter algorithm.

In another aspect of the present disclosure, at least one of the first algorithm or the second algorithm is based on a poral valve model or on a decomposition model wherein the sensed data is decomposed into a tonic component, a phasic component, and a noise component.

In yet another aspect of the present disclosure, the first algorithm includes at least one neural network. More specifically, in aspects, a first neural network of the at least one neural network may be configured to model how the estimated nervous system state at least one of: evolves with time or relates to observations, and a second neural network of the at least one neural network may be configured to estimate the nervous system state. Additionally or alternatively, the computing device may be configured to re-train the at least one neural network and include updated neural network weights in the updates provided from the computing device to the processing device.

In still another aspect of the present disclosure, the system further includes at least one second sensor configured to sense a second continuously variable non-neural physiological condition and to output second sensed data indicative of the second continuously variable non-neural physiological condition, wherein the first algorithm is configured to estimate the state of the nervous system based on the sensed data and the second sensed data. In such aspects, the first and second continuously variable non-neural physiological conditions may be skin conductance at different bodily locations. Alternatively, the first continuously variable non-neural physiological condition may be skin conductance and/or the second continuously variable non-neural physiological condition may be heart rate.

A control system provided in accordance with the present disclosure includes the system according to any of the aspects above or otherwise detailed herein. The control system further includes at least one of: a therapy-providing device configured to receive the estimated state of the nervous system output from the processing device and to provide a therapy to a user based thereon; or an output device configured to receive the estimated state of the nervous system output from the processing device and to provide an output based thereon.

A method for estimating a state of the nervous system in accordance with aspects of the present disclosure includes: obtaining sensed data indicative of a continuously variable non-neural physiological condition; estimating, using a relatively low performance device, a state of a nervous system based on the sensed data; outputting the estimated state of the nervous system; and receiving, at the relatively low performance device, updates from a relatively high performance device to improve the estimating.

In an aspect of the present disclosure, obtaining the sensed data includes obtaining skin conductance data from at least one skin conductance sensor, and estimating the state of the nervous system includes: an identification of an autonomic nervous system activation estimating a state of sympathetic arousal.

In another aspect of the present disclosure, obtaining the sensed data includes obtaining blood cortisol level data, and estimating the state of the nervous system includes estimating a state of cortisol-related energy production.

In still another aspect of the present disclosure, the method further includes receiving an external input. In such aspects, the estimating is based on the sensed data and the external input.

In yet another aspect of the present disclosure, the method further includes estimating, at the relatively high performance device, the state of the nervous system based on the sensed data and determining updated parameters based upon the estimating. The updates include the updated parameters.

In still yet another aspect of the present disclosure, the estimating is performed in real-time and the updates are received occasionally.

In an aspect of the present disclosure, the estimating includes a sparse recovery algorithm and the updates are based at least partially on a sparse recovery algorithm and one of: an expectation maximization algorithm or a coordinate descent algorithm. In such aspects, the sparse recovery algorithm includes a least squares algorithm or a Bayesian filter algorithm.

In another aspect of the present disclosure, at least one of the estimating or the updates are based on a poral valve model or a decomposition model wherein the sensed data is decomposed into a tonic component, a phasic component, and a noise component.

In another aspect of the present disclosure, the estimating includes running a forward filter algorithm, and the updates include updated model parameters for the forward filter algorithm. In aspects, the updated model parameters are determined by repeatedly running the forward filter algorithm and a backward smoothing algorithm.

In yet another aspect of the present disclosure, the estimating includes running a first neural network to model how the estimated nervous system state at least one of: evolves with time or relates to observations, and running a second neural network to estimate the nervous system state. In such aspects, the updates may include updated neural network weights, which may be determined by re-training the at least one neural network.

In still another aspect of the present disclosure, the method further includes obtaining second sensed data indicative of a second continuously variable non-neural physiological condition. In such aspects, the estimating is based on the sensed data and the second sensed data. The first and second continuously variable non-neural physiological conditions may be skin conductance at different bodily locations or may be measures of different physiological conditions.

A method of control in accordance with the present disclosure includes the method according to any of the aspects above or otherwise herein, and further includes receiving the estimated state of the nervous system; and at least one of: providing a therapy to a user based on the received estimated state of the nervous system; or providing an output based on the received estimated state of the nervous system.

### BRIEF DESCRIPTION OF THE DRAWINGS

Various aspects and features of the present disclosure are described hereinbelow with reference to the drawings wherein:

FIG. 1A is an illustration of a system for estimating a nervous system state based on measurement of a physiological condition in accordance with the present disclosure;

FIG. 1B is a block diagram of the system of FIG. 1A;

FIG. 2A is a block diagram of one implementation of the system of FIG. 1A;

FIG. 2B is a block diagram of another implementation of the system of FIG. 1A;

FIG. 3 is a flow diagram illustrating a method of estimating a nervous system state based on measurement of a physiological condition in accordance with the present disclosure;

FIG. 4 is a flow diagram illustrating another method of estimating a nervous system state based on measurement of a physiological condition in accordance with the present disclosure;

FIG. 5A is an illustration of another system for estimating a nervous system state based on measurements of physiological conditions in accordance with the present disclosure;

FIG. 5B is a block diagram of one implementation of the system of FIG. 5A;

FIG. 6A is a flow diagram illustrating a calibration method of the present disclosure;

FIG. 6B is a flow diagram of still another method of estimating a nervous system state based on measurements of multiple physiological conditions in accordance with the present disclosure;

FIG. 7A is a control system in accordance with the present disclosure for regulating the nervous system based on an estimated nervous system state;

FIG. 7B is another control system in accordance with the present disclosure for communication with an additional device(s) and/or system(s) based on an estimated nervous system state;

FIGS. 8A and 8B are illustrations of single-channel and multi-channel systems, respectively, for identifying autonomic nervous system activations based on measurement(s) of a physiological condition in accordance with the present disclosure;

FIG. 9 is a block diagram of a hardware configuration configured for use with the systems of FIGS. 8A and/or 8B in accordance with the present disclosure;

FIG. 10 is a series of anatomical illustrations of the stages of the poral valve model in accordance with the present disclosure; and

FIG. 11 is an illustrated flow diagram for identifying autonomic nervous system activations based on measurement(s) of a physiological condition in accordance with the present disclosure.

### DETAILED DESCRIPTION

Systems and methods of the present disclosure enable the estimation of a nervous system state based on a measurement(s) of a continuously variable, non-neural physiological condition(s). For example, as the skin contains sweat glands that are innervated by nerve fibers, analyzing changes in the conductance of the sweat secretions on the skin, e.g., skin conductance, and/or other physiological properties or parameters of sweat secretions on the skin can be utilized to infer a state of sympathetic arousal of the autonomic nervous system (e.g., by identifying autonomic nervous system activations). As another example, analyzing changes of cortisol levels in the blood can be utilized to infer a state of cortisol-related energy production.

The present disclosure further provides, in aspects, control systems, e.g., closed-loop or open-loop controllers, that enable the regulation of the nervous system based on the estimated nervous system state, thereby enabling treatment of, for example, disorders and conditions that may result from the nervous system deviating from its usual range of functioning. More specifically, as sympathetic arousal is closely related to emotional arousal (the level of activation or excitement accompanying an emotion), estimates of sympathetic arousal may be utilized as part of a control system to treat emotional disorders relating to abnormal levels of arousal, e.g., by providing therapies that reduce or increase sympathetic arousal as needed. Likewise, estimates of cortisol-related energy production can be utilized to provide cortisol regulating medicament (or other therapies), e.g., via an automated infusion pump or a recommendation to give a manual dose, to treat disorders related to either too much or too little cortisol being secreted into the bloodstream.

Although the aspects and features of the present disclosure are detailed hereinbelow with respect to detecting and analyzing skin conductance and cortisol levels to estimate sympathetic arousal and cortisol-related energy production, respectively, the aspects and features of the present disclosure are likewise applicable for use with measurements of additional or alternative continuously variable, non-neural physiological conditions for estimating states of the nervous system or other physiological systems.

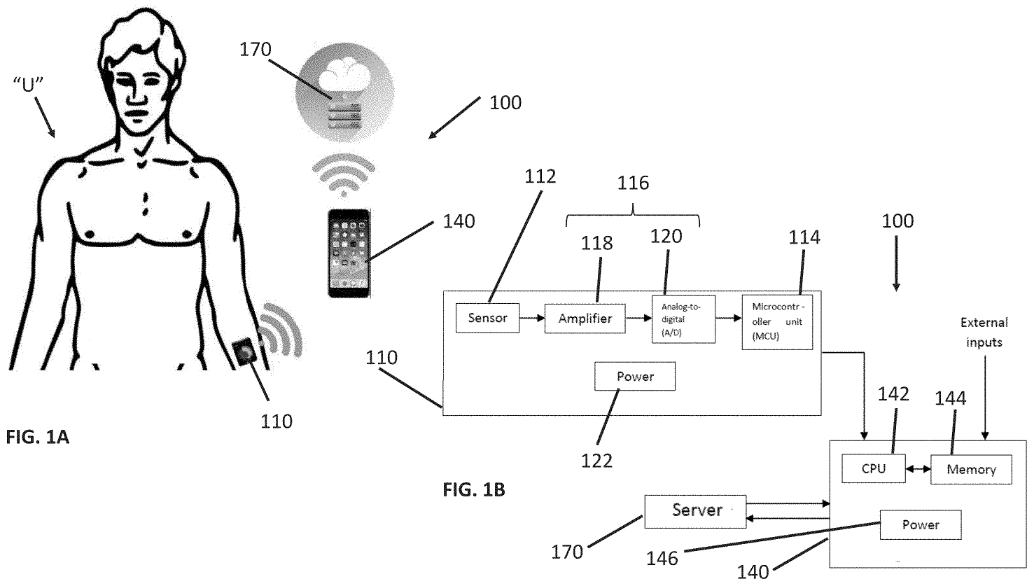

Turning to FIGS. 1A and 1B, a system 100 provided in accordance with the present disclosure for estimating a nervous system state based on measurement of a (non-neural) physiological condition includes a sensor assembly 110, a processing device 140, and a server 170. Sensor assembly 110 may be embodied as or incorporated into: a wearable article of clothing of piece of jewelry, e.g., a watch, bracelet, ring, anklet, necklace, sock, glove, wristband, etc., to be worn by a user “U;” a device configured to be attached to the user “U” for an extended period of time yet removable therefrom, e.g., a patch, a chest strap, ear piece, band, brace, etc.; or a device configured to be periodically or intermittently coupled to the user “U” to obtain measurement data, e.g., a probe, cuff, pen, etc.

Regardless of the particular configuration of sensor assembly 110, sensor assembly 110 generally includes: a sensor 112 configured to measure a physiological condition, e.g., skin conductance, blood cortisol level, heart rate, etc., and to generate a signal in response thereto; a microcontroller 114, e.g., including a central processing unit (CPU) and a memory storing instructions to be executed by the CPU, configured to receive the signal from sensor 112 and process to same to generate physiological condition data, e.g., a skin conductance value, a blood cortisol level, etc., based on the signal.

Sensor assembly 110 may further include intermediate circuitry 116 such as, for example, an amplifier 118, an analog-to-digital (A/D) converter 120, and/or other suitable circuitry operably coupled between the sensor 112 and microcontroller 114 to convert, amplify, or otherwise the signal generated by sensor 112 for input to microcontroller 114. Sensor assembly 110 also includes a power source 122, e.g., a battery, configured to power sensor assembly 110. Alternatively, sensor assembly 110 may be configured to connect to an external power source, e.g., a wall outlet.

Sensor 112, as noted above, may be configured to measure skin conductance, blood cortisol level, etc. With regard to skin conductance, sensor 112 may be a galvanic skin response (GSR) sensor or other suitable sensor configured to sense the conductance of sweat secretions on the skin. With respect to blood cortisol levels, sensor 112 may include a cortisol-sensitive membrane to enable blood cortisol levels to be determined from sweat secretions on the skin, may be a surface plasmon resonance (SPR) sensor configured to optically sense blood cortisol levels, or may be any other suitable sensor configured to sense blood cortisol levels.

Microcontroller 114 is configured to output the physiological condition data to processing device 140. The memory of microcontroller 114 may be a volatile type memory, e.g., RAM, or a non-volatile type memory, e.g., flash media, disk media, etc. Further, as an alternative to a CPU, microcontroller 114 may include any other suitable processor(s) such as, for example, a digital signal processor, a microprocessor, an ASIC, a graphics processing unit (GPU), field-programmable gate array (FPGA), etc.

Processing device 140 may be partially or wholly integrated into sensor assembly 110 or may be separate therefrom and operably coupled to sensor assembly 110 via a wired or wireless connection, directly or indirectly. Processing device 140 may be embodied in a single device or incorporated across multiple devices. Processing device 140 may be embodied as or incorporated at least partially into a server, computer, smartphone, tablet, combinations thereof, etc. Processing device 140 generally includes a CPU 142, a memory 144 storing instructions to be executed by the CPU 144, and a power source 146, e.g., a battery. As an alternative or in addition to CPU 142, any other suitable processor(s) may be utilized such as, for example, a digital signal processor, a microprocessor, an ASIC, a graphics processing unit (GPU), field-programmable gate array (FPGA), etc. Memory 144 may include one or more of a volatile type memory, e.g., RAM, or a non-volatile type memory, e.g., flash media, disk media, etc. As an alternative to battery 146, processing device 140 may be configured to connect to an external power source, e.g., a wall outlet. As utilized herein, the terms “device,” “server,” and the like need not be embodied in a single hardware unit but may instead include virtual devices, servers, etc., and/or devices, servers, etc. embodied across multiple hardware units.

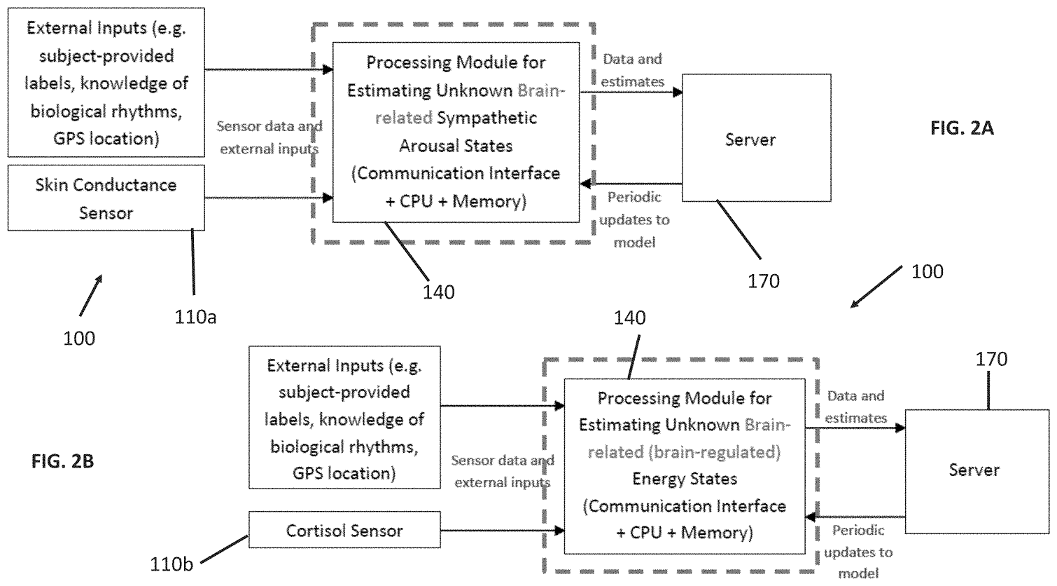

With additional reference to FIGS. 2A and 2B, processing device 140, as noted above, is configured to receive the physiological condition data from sensor assembly 110 (e.g., a skin conductance sensor assembly 110a or a cortisol sensor assembly 110b). Processing device 140 stores a program, e.g., including one or more algorithms, configured to estimate a nervous system state based on the physiological condition data and, if provided, additional input data. More specifically, where a skin conductance sensor assembly 110a is utilized, as shown in FIG. 2A, the program of processing device 140 is configured to consider the skin conductance data along with any additional input data (e.g., from external inputs) and, based thereon, estimate a state of sympathetic arousal. Where a cortisol sensor assembly 110b is utilized, as shown in FIG. 2BA, the program of processing device 140 is configured to consider the cortisol level data along with any additional input data (e.g., from external inputs) and, based thereon, estimate a state of cortisol-related energy production.

As noted above, processing device 140 may be configured to receive additional input data (e.g., from external inputs). More specifically, in such aspects, processing device 140 is configured to communicate with one or more external devices to receive the additional input data and to enable updating of the stored program (and/or algorithm(s) thereof). With regard to this additional input data, processing device 140 may be configured to receive, for example: demographic information (age, height/weight, sex, ethnicity, etc.), medical history information, historical nervous system state information, user-provided labels (e.g., relating to emotional feeling, energy level, etc.), user-provided indications/symptoms, healthcare professional-provided labels, healthcare professional-provided indications/symptoms, historical and/or contemporaneous physiological condition data (including biological rhythm data, heart rate data, etc.), environmental data (including, for example, GPS location data, motion data, temperature data, and/or time of day data, from which other information such as a relative noise level can be determined), and/or other suitable data. Some or all of this additional input data may be utilized via processing device 140 and/or server 170, together with the physiological condition data received from sensor assembly 100, to facilitate estimation of the nervous system state.

Server 170 may be, for example, a cloud server or other suitable server or group of servers configured to communicate with processing device 140. Server 170, more specifically, may be configured to receive the estimated nervous system state (and, in configurations, the physiological condition data and/or the additional input data) from processing device 140 to enable updating of the program (and/or algorithm(s) thereof) of processing device 140, e.g., updating the model parameters, adjusting the weights thereof, etc. In aspects, server 170 communicates with the processing devices 140 of plural systems 100 to enable use of data and information from plural systems 100 to facilitate updating the model parameters, weights, etc., for the program of each processing device 140.

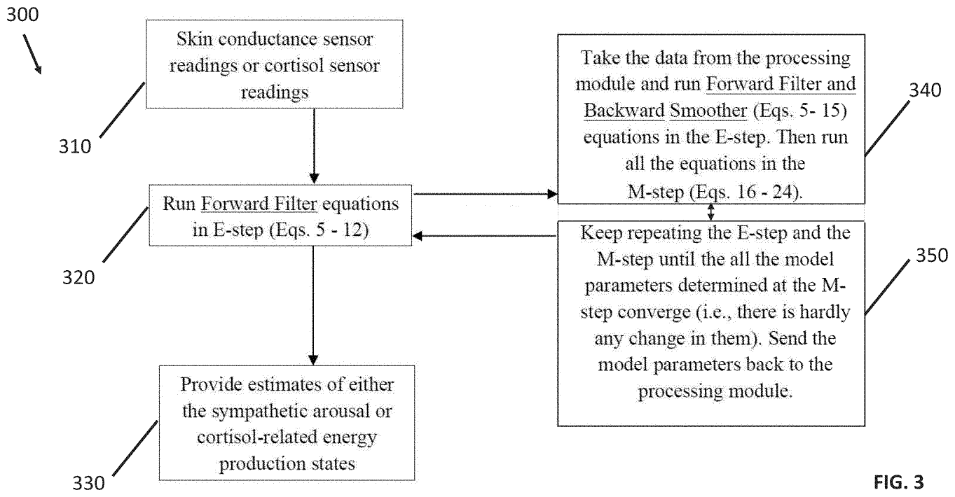

Referring to FIG. 3, a method 300 is provided in accordance with the present disclosure, e.g., to be implemented by system 100 (FIGS. 1A-2B), for estimating a nervous system state, e.g., a state of sympathetic arousal or a state of cortisol-related energy production, based on physiological condition data, e.g., skin conductance data or cortisol level data, respectively.

Method 300 involves the use of an expectation-maximization (EM) framework employing Bayesian filtering (e.g., implemented using iteratively-reweighted Kalman filtering and backward smoothing), as detailed below. Method 300 may be utilized to provide real-time estimates of a nervous system state to a user “U” (FIG. 1A), e.g., via a visual display, to provide real-time nervous system state feedback as part of a control system that regulates, for example, sympathetic arousal or cortisol-related energy production, and/or to provide outputs to other devices and systems, e.g., Internet of Things (IoT) devices, healthcare provider device(s), caregiver device(s), etc. in accordance with the estimated nervous system state.

Method 300, more specifically, estimates an unknown nervous system state e.g., a state of sympathetic arousal or cortisol-related energy production, denoted x_{κ}, that is assumed to vary with time according to Equation (1):

xₖ=xₖ₋₁+εₖ

wherein ε_{κ} is noise and wherein x_{κ} is taken as related to a continuous variable s_{κ} according to Equation (2):

sₖ=δ₀+δ₁xₖ+wₖ

wherein w_{κ} is noise and δ₀ and δ₁ are model parameters.

Using n_{κ}=1 or n_{κ}=0 to denote whether a point process event (e.g., a sequence of point process events with certain amplitudes) occurred, x_{κ} is taken as related to the point process event occurrence probability p_{κ} and the correspondence amplitude r_{κ} through Equation (3):

$${p}_{k} = \frac{1}{1 + {e}^{- ( {β}_{0} + {β}_{1} {x}_{k} )}}$$

### and Equation (4):

rₖ=γ₀+γ₁xₖ+vₖ

wherein v_{κ} is noise and wherein γ₀, γ₁, β₀, and β₁ are model parameters.

Initially, at 310, the physiological condition data, e.g., skin conductance data or cortisol level data, is input to a forward filter portion of the EM framework. The forward filter portion, as indicated at 320, estimates a nervous system state, x_{κ}, e.g., a state of sympathetic arousal or cortisol-related energy production, based on the input physiological condition data using Equations (5)-(12), below.

x_{k|k-1}=x_{k-1|k-1} Equation (5):

Equation (6):

$$\begin{matrix} {σ}_{k \vert k - 1}^{2} = {σ}_{k - 1 \vert k - 1}^{2} + {σ}_{ɛ}^{2} \\ If {n}_{k} = 0 , & \\ {C}_{k} = \frac{{σ}_{k \vert k - 1}^{2}}{{δ}_{1}^{2} {σ}_{k \vert k - 1}^{2} + {σ}_{w}^{2}} & Equation ( 7 ) \\ {x}_{k \vert k} = {x}_{k \vert k - 1} + {C}_{k} [ {β}_{1} {σ}_{w}^{2} ( {n}_{k} - {p}_{k \vert k} ) + {δ}_{1} ( {s}_{k} - {δ}_{0} - {δ}_{1} {x}_{k \vert k - 1} ) ] & Equation ( 8 ) \\ and {σ}_{k \vert k}^{2} = {[ \frac{1}{{σ}_{k \vert k}^{2}} + {β}_{1}^{2} {p}_{k \vert k} ( 1 - {p}_{k \vert k} ) + \frac{{δ}_{1}^{2}}{{σ}_{w}^{2}} ]}^{- 1} & Equation ( 9 )\end{matrix}$$

If n_{κ}=1, Equation (10):

$$\begin{matrix} {C}_{k} = \frac{{σ}_{k \vert k - 1}^{2}}{{σ}_{v}^{2} {σ}_{w}^{2} + {σ}_{k \vert k - 1}^{2} ( {γ}_{1}^{2} {σ}_{w}^{2} + {δ}_{1}^{2} {σ}_{v}^{2} )} & \\ and {x}_{k \vert k} = {x}_{k \vert k - 1} + {C}_{k} [ {β}_{1} {σ}_{v}^{2} {σ}_{w}^{2} ( {n}_{k} - {p}_{k \vert k} ) + {γ}_{1} {σ}_{w}^{2} ( {r}_{k} - {γ}_{0} - {γ}_{1} {x}_{k \vert k - 1} ) + {δ}_{1} {σ}_{v}^{2} ( {s}_{k} - {δ}_{0} - {δ}_{1} {x}_{k \vert k - 1} ) ] & Equation ( 11 ) \\ Then , {σ}_{k \vert k}^{2} = {[ \frac{1}{{σ}_{k \vert k - 1}^{2}} + {β}_{1}^{2} {p}_{k \vert k} ( 1 - {p}_{k \vert k} ) - \frac{{γ}_{1}^{2}}{{σ}_{v}^{2}} + \frac{{δ}_{1}^{2}}{{σ}_{w}^{2}} ]}^{- 1} & Equation ( 12 )\end{matrix}$$

The estimated nervous system state, e.g., state of sympathetic arousal or cortisol-related energy production, is then output at 330. The above, namely 310-330, may be performed via processing device 140 of system 100 (FIGS. 1A-2B) or any other suitable processing device(s) in any other suitable system. 310-330 may be performed continuously to provide real-time estimation as to the estimated nervous system state.

Intermittently or periodically (e.g., every 30 minutes) during method 300, the input physiological condition data is utilized to run the forward filter portion of the EM framework together with a backward smoother portion of the EM framework, collectively the expectation portion (E-step) of the EM framework, as indicated in the first part of 340. The forward filter portion is detailed above with respect to Equations (5)-(12). The backward smoother portion is utilized to obtain final x_{κ} estimates by proceeding in the backward direction using Equations (13)-(15), below.

$$\begin{matrix}{A}_{k} = \frac{{σ}_{k \vert k}^{2}}{{σ}_{k + 1 \vert k}^{2}} & Equation ( 13 ) \\ {x}_{k \vert k} = {x}_{k \vert k} + {A}_{k} ( {x}_{k + 1 \vert K} - {x}_{k + 1 \vert k} ) & Equation ( 14 ) \\ {σ}_{k \vert K}^{2} = {σ}_{k \vert k} + {A}_{k}^{2} ( {σ}_{k + 1 \vert K}^{2} - {σ}_{k + 1 \vert k}^{2} ) & Equation ( 15 )\end{matrix}$$

The maximization portion (M-step) of the EM framework is then performed, as indicated in the second part of 340. The M-step, more specifically, is performed according to Equations (16)-(24), below.

$$\begin{matrix} {U}_{k} = {x}_{k \vert k}^{2} + {σ}_{k \vert k}^{2} & Equation ( 16 ) \\ {U}_{k , k + 1} = {x}_{k \vert K} {x}_{k + 1 \vert K} + {A}_{k} {σ}_{k + 1 \vert K}^{2} & Equation ( 17 ) \\ [ \begin{matrix}{γ}_{0}^{( l + 1 )} \\ {γ}_{1}^{( l + 1 )}\end{matrix} ] = {[ \begin{matrix}\vert \tilde{K} \vert & {\sum }_{k ∈ \tilde{K}} {x}_{k \vert K} \\ {\sum }_{k ∈ \tilde{K}} {x}_{k \vert K} & {\sum }_{k ∈ \tilde{K}} {U}_{k}\end{matrix} ]}^{- 1} [ \begin{matrix}{\sum }_{k ∈ \tilde{K}} {r}_{k} \\ {\sum }_{k ∈ \tilde{K}} {r}_{k} {x}_{k \vert K}\end{matrix} ] & Equation ( 18 ) \\ [ \begin{matrix}{δ}_{0}^{( l + 1 )} \\ {δ}_{1}^{( l + 1 )}\end{matrix} ] = {[ \begin{matrix}K & {{\sum }_{k = 2}}^{K} {x}_{k \vert K} \\ {{\sum }_{k = 2}}^{K} {x}_{k \vert K} & {{\sum }_{k = 2}}^{K} {U}_{k}\end{matrix} ]}^{- 1} [ \begin{matrix}{{\sum }_{k = 2}}^{K} {s}_{k} \\ {{\sum }_{k = 2}}^{K} {s}_{k} {x}_{k \vert K}\end{matrix} ] & Equation ( 19 ) \\ {σ}_{v}^{2 ( l + 1 )} = \frac{1}{\vert \tilde{K} \vert} [ {\sum }_{k ∈ \tilde{K}} {r}_{k}^{2} + \vert \tilde{K} \vert {γ}_{0}^{2 ( l + 1 )} + {γ}_{1}^{2 ( l + 1 )} {\sum }_{k ∈ \tilde{K}} {U}_{k} - 2 {γ}_{0}^{( l + 1 )} {\sum }_{k ∈ \tilde{K}} {r}_{k} - 2 {γ}_{1}^{( l + 1 )} {\sum }_{k ∈ \tilde{K}} {x}_{k \vert K} {r}_{k} + 2 {γ}_{0}^{( l + 1 )} {γ}_{1}^{( l + 1 )} {\sum }_{k ∈ \tilde{K}} {U}_{k} ] & Equation ( 20 ) \\ {σ}_{w}^{2 ( l + 1 )} = \frac{1}{K} [ {{\sum }_{k = 1}}^{K} {s}_{k}^{2} + K {δ}_{0}^{2 ( l + 1 )} + {δ}_{1}^{2 ( l + 1 )} {{\sum }_{k = 1}}^{K} {U}_{k} - 2 {δ}_{0}^{( l + 1 )} {{\sum }_{k = 1}}^{K} {s}_{k} - 2 {δ}_{1}^{( l + 1 )} {{\sum }_{k = 1}}^{K} {x}_{k \vert K} {s}_{k} + 2 {δ}_{0}^{( l + 1 )} {δ}_{1}^{( l + 1 )} {{\sum }_{k = 1}}^{K} {U}_{k} ] & Equation ( 21 ) \\ {σ}_{ɛ}^{2 ( l + 1 )} = \frac{1}{K} [ {{\sum }_{k = 2}}^{K} {U}_{k} - 2 {{\sum }_{k = 1}}^{K - 1} {U}_{k , k + 1} + {{\sum }_{k = 1}}^{K - 1} {U}_{k} ] & Equation ( 22 ) \\ {\sum }_{k = 1}^{K} [ {n}_{k} - {p}_{k \vert K} - \frac{1}{2} {β}_{1}^{2 ( l + 1 )} {σ}_{k \vert K}^{2} ( 1 - {p}_{k \vert K} ) ( 1 - 2 {p}_{k \vert K} ) {p}_{k \vert K} ] = 0 & Equation ( 23 ) \\ {\sum }_{k = 1}^{K} \{ {n}_{k} {x}_{k \vert K} - {x}_{k \vert K} {p}_{k \vert K} - {β}_{1}^{( l + 1 )} {p}_{k \vert K} ( 1 - {p}_{k \vert K} ) [ 2 + {β}_{1}^{( l + 1 )} {x}_{k \vert K} ( 1 - 2 {p}_{k \vert K} ) ] \} = 0 & Equation ( 24 )\end{matrix}$$

The EM framework indicated at 340 is repeated as necessary until the M-step results sufficiently converge such that there is minimal change in the result from run to run, as indicated at 350. Once suitable convergence has been achieved, the resultant model parameters are stored and/or output for subsequent use in the forward filter portion indicated at 320.

The above, namely 340 and 350, may be performed via server 170 of system 100 (FIGS. 1A-2B), with the results being fed back to processing device 140 of system 100 (FIGS. 1A-2B) to update the model parameters of the program (and/or algorithm(s) thereof) of processing device 140. Alternatively, 310-350 may be performed all within processing device 140, all within server 170, via different combinations thereof, and/or utilizing additional or alternative devices. As can be appreciated, updating the model parameters improves the estimated nervous system state, e.g., state of sympathetic arousal or cortisol-related energy production, output at 330.

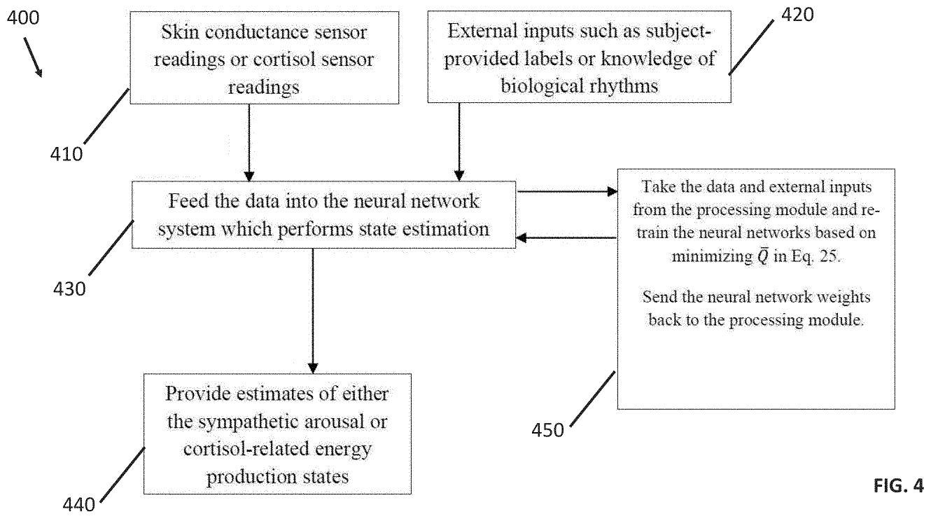

With reference to FIG. 4, another method 400 is provided in accordance with the present disclosure, e.g., to be implemented by system 100 (FIGS. 1A-2B), for estimating nervous system state, e.g., a state of sympathetic arousal or a state of cortisol-related energy production, based on physiological condition data, e.g., skin conductance data or cortisol level data, respectively, and additional input data. Method 400, as detailed below, involves the use of a neural network system including one or more neural networks, e.g., first and second neural networks, that are trained in such a way that an external influence (e.g., the additional input data) is utilized; the extent to which the external influence affects the estimated nervous system state can be modified by tuning certain parameters of the system, as detailed below. Method 400 may be utilized to provide real-time estimates of a nervous system state to a user “U” (FIG. 1A), e.g., via a visual display, to provide real-time nervous system state feedback as part of a control system that regulates, for example, sympathetic arousal or cortisol-related energy production, and/or to provide outputs to other devices and systems, e.g., Internet of Things (IoT) devices, healthcare provider device(s), caregiver device(s), etc. in accordance with the estimated nervous system state.

Method 400, more specifically, may employ a neural network system including two neural networks, e.g., a first neural network to model how the state evolves with time and how it relates to observations, and a second neural network to estimate the state. The two neural networks are trained by minimizing Equation (25):

Q̅=(1−ρ)Q+ρΣ(xₖ−lₖ)²

wherein, according to Equation (26):

$$Q = - \{ {\sum }_{k = 1}^{K} {𝔼}_{{q}_{ϕ} ( {x}_{k} \vert \vec{{y}_{k}} )} [ log {p}_{ψ} ( {y}_{k} \vert {x}_{k} ) ] - KL ( {q}_{ϕ} ( {x}_{1} \vert \vec{{y}_{k}} ) ‖ {p}_{ψ} ( {x}_{1} ) ) - {\sum }_{k = 1}^{K} {𝔼}_{{q}_{ϕ} ( {x}_{k} \vert \vec{{y}_{k}} )} [ KL ( {q}_{ϕ} ( {x}_{k - 1} , \vec{{y}_{k}} ) ‖ {p}_{ψ} ( {x}_{k} \vert {x}_{k - 1} ) ) ] \}$$

As above, x_{κ} is the unknown nervous system state e.g., a state of sympathetic arousal or cortisol-related energy production; y_{κ} consists of the observations n_{κ}, r_{κ}, and s_{κ}; p and q denote probability density functions; and ψ and ϕ denote the weights of the first neural network. Further, l_{κ} is the external influence and p is constant between 0 and 1 depending upon how much the external influence is permitted to affect the state estimate x_{κ}.

Different configurations for either or both of the neural networks, e.g., different numbers of layers, numbers of neurons in each layer, etc., may be utilized depending upon the particular application, e.g., whether a state of sympathetic arousal is estimated based on skin conductance data or whether a state of cortisol-related energy production is estimated based on cortisol level data.

As indicated at 410 and 420 of FIG. 4, the physiological condition data, e.g., skin conductance data or cortisol level data, and the external input(s), respectively, are obtained and are input to the neural network system to perform state estimation, as indicated at 430. The neural network system, based on the state estimation performed, outputs an estimation of the state, as indicated at 440. 410-440 may be performed via processing device 140 of system 100 (FIGS. 1A-2B) or any other suitable processing device(s) in any other suitable system. The external input(s) may include some or all of the additional input data noted above. In particular, the external input may include location data (e.g., from a GPS system), to enable the neural network system to consider whether the user is in a low noise environment (e.g., a workplace office) or a high noise environment (e.g., a gym). Biological rhythm data may also be utilized. User labels (e.g., how the user is feeling emotionally, whether the user is tired or energetic, etc.) may additionally or alternatively be utilized as part of the external input.

Intermittently or periodically during method 400, the estimated state and/or the input data is utilized to re-train the neural network system and determine (if necessary) modified weights ψ and ϕ, as indicated at 450, that are fed back to the neural network system to improve accuracy of the estimated nervous system state output at 440. The frequency of this re-training may depend, for example, on the external inputs utilized. That is, where the external input includes information relating to biological rhythm, for example, re-training may be performed less frequently (e.g., weekly or monthly), since this information is not likely to change quickly. On the other hand, where the external inputs are labels provided by the user, as another example, re-training may be performed more often (e.g., every 30 minutes) such that the inputs are taken into consideration more quickly when providing the estimated nervous system state. In still another example, re-training may occur even more frequently or intermittently on occurrence of an event, e.g., the user moving from a low noise environment to a high noise environment (where environment noise information is provided as an external input), to enable adjustment of the weights accordingly.

The neural network system may include a convolutional neural network (CNN), a recurrent adversarial network (RAN), a generative adversarial network (GAN) and/or other suitable neural networks. As an alternative or in addition to a neural network, other suitable machine learning systems may be utilized such as, for example a support vector machine (SVM), and/or may implement: Bayesian Regression, Naive Bayes, nearest neighbors, least squares, means, and support vector regression, among other data science and artificial science techniques.

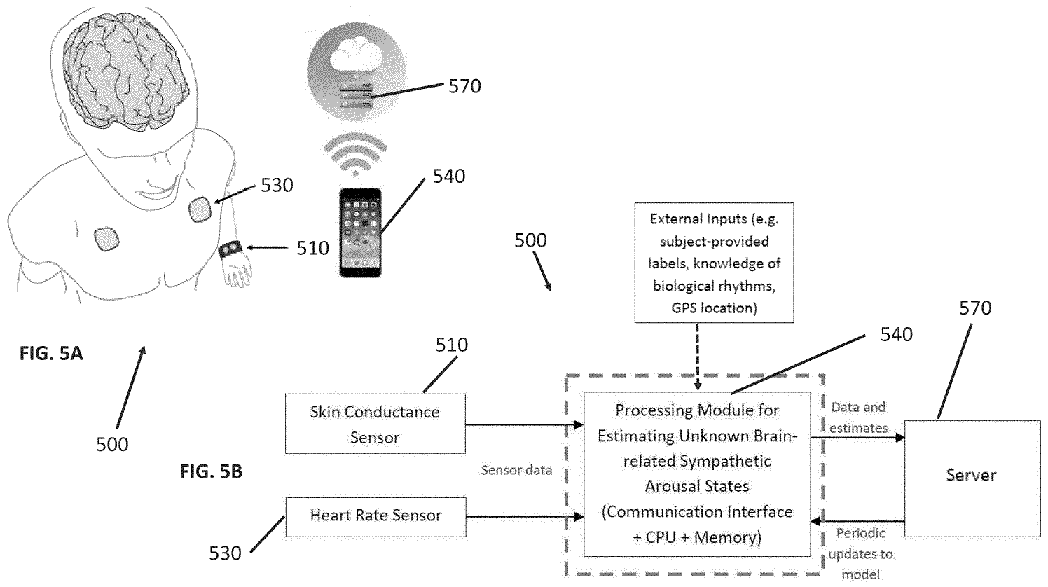

Referring to FIGS. 5A and 5B, another system 500 provided in accordance with the present disclosure for estimating a nervous system state, e.g., sympathetic arousal, based on measurement of (non-neural) physiological conditions includes a first sensor assembly 510 (e.g., a skin conductance sensor), a second sensor assembly 530 (e.g., a heart rate sensor such as an EKG monitor or other suitable heart rate monitor), a processing device 540, and a server 570. System 500 is similar to and may include any of the features of system 100 (FIGS. 1A-2B), detailed above, except that system 500 utilizes two continuously variable (non-neural) physiological conditions as inputs: skin conductivity and heart rate (although additional or alternative physiological condition inputs are also contemplated). Thus, for the purposes of brevity, only differences between system 500 and system 100 (FIGS. 1A-2B) are described in detail below while similarities are summarily described or omitted entirely. Further, system 500 may utilize method 300 (FIG. 3), method 400 (FIG. 4), or any other suitable method(s).

Processing device 540 is configured to receive the physiological condition data from each of first and second sensor assemblies 510, 530 and utilizing a program, e.g., including one or more algorithms, stored thereon, is configured to estimate a nervous system state based on the physiological condition data and, if provided, additional input data. Server 570 is configured to receive the estimated nervous system state (and, in configurations, the physiological condition data and/or the additional input data) from processing device 540 to enable updating of the program (and/or algorithm(s) thereof) of processing device 540, e.g., updating the model parameters thereof, adjustment of the weights thereof, etc., to facilitate estimating the nervous system state, e.g., sympathetic arousal.

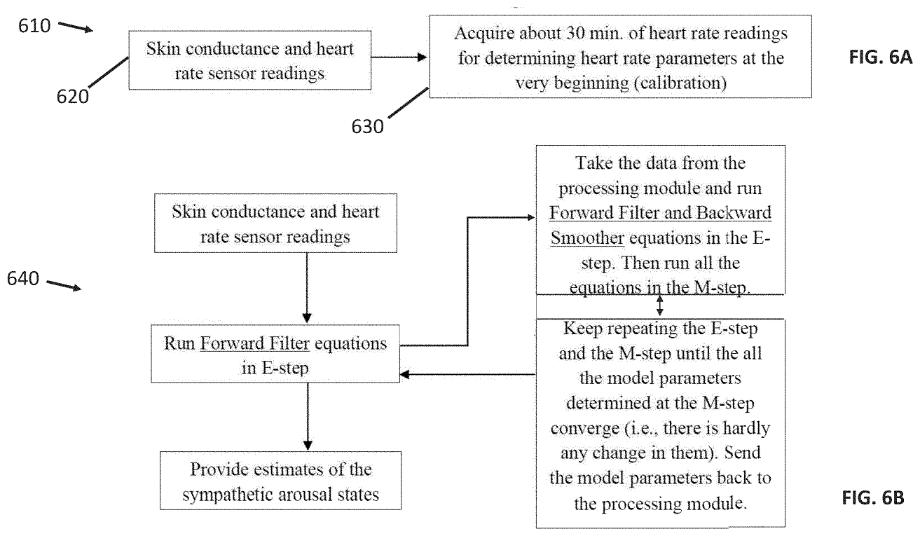

With reference to FIGS. 6A and 6B, in conjunction with FIGS. 5A and 5B, a method is provided in accordance with the present disclosure, e.g., to be implemented by system 500, for estimating a nervous system state, e.g., a state of sympathetic arousal, based on data regarding at least two physiological conditions, e.g., skin conductance data and heart rate data. Referring first to FIG. 6A, the method initially includes a calibration portion 610, wherein the skin conductance data and heart rate data are collected for an initial period, as indicated at 620, to enable determination of heart rate parameters and system calibration, as indicated at 630.

Turning to FIG. 6B, once calibration is complete, the method proceeds to an estimation portion 640. Estimation portion 640 is similar to method 300 (FIG. 3), and may be implemented as detailed above with respect to method 300 (FIG. 3), except that both skin conductance data and heart rate data are input in order to estimate the state of sympathetic arousal.

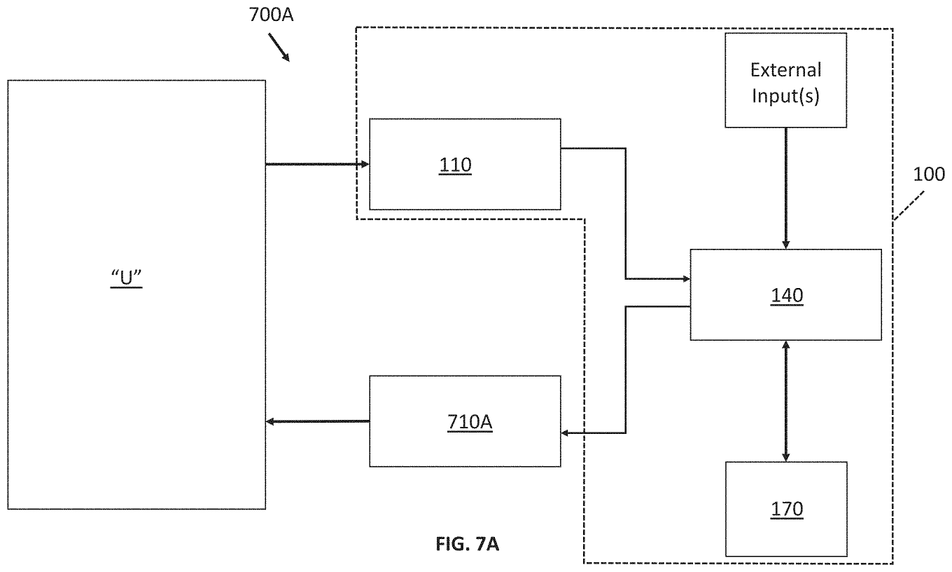

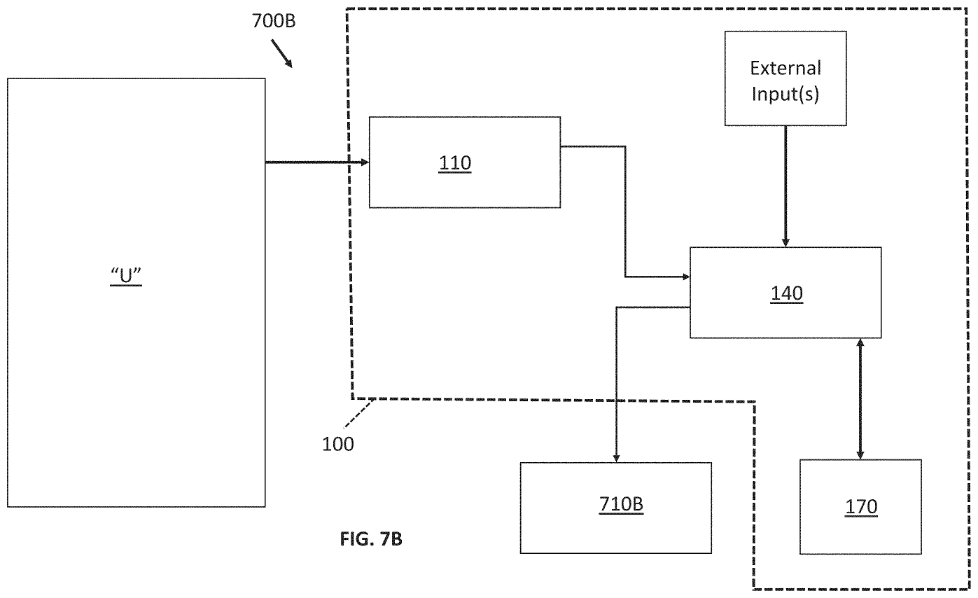

FIGS. 7A and 7B illustrates systems 700A, 700B, respectively, provided in accordance with the present disclosure. Systems 700A and 700B provide a closed-loop control system and an open-loop control system, respectively, to enable or facilitate the regulation of the nervous system based on an estimated nervous system state, e.g., sympathetic arousal or cortisol-related energy production.

Referring in particular to FIG. 7A, system 700A incorporates system 100 (see also FIGS. 1A-2B) and, more specifically, sensor assembly 110, processing device 140, and server 170 of system 100. Alternatively, system 500 (FIGS. 5A-5B) may be utilized. System 700A further includes a therapy-providing device 710A such as, for example, an infusion pump, an injection pen, a pharmaceutical patch, an inhaler, or other suitable therapy-providing device. However, the therapy is not limited to pharmaceutical therapies but, rather, other therapies are also contemplated such as, for example, such as stimulation therapy (via a stimulation device), visual and/or audio therapy (via a suitable visual and/or audio device), etc. Therapy-providing device 710A is configured to provide therapy, e.g., automatically or via instructions to the user “U” for manual delivery, as necessary based on the estimated nervous system state provided by system 100, thereby providing closed-loop regulation of the nervous system state, e.g., sympathetic arousal or cortisol-related energy production, according to the estimated state provided by system 100. Sensor assembly 110 and/or processing device 140 of system 100 may be integrated with therapy-providing device 710A or separate therefrom. In separate configurations, processing device 140 is configured to communicate with therapy-providing device 710A via wired or wireless connection, directly or indirectly.

With reference to FIG. 7B, system 700B incorporates system 100 (see also FIGS. 1A-2B) and, more specifically, sensor assembly 110, processing device 140, and server 170 of system 100. Alternatively, system 500 (FIGS. 5A-5B) may be utilized. System 700B further includes an output device 710B such as, for example, a communications device (of the user “U,” a healthcare provider, a caregiver, etc.), an Internet of Things (IoT) device such as a smart appliance (e.g., lighting system, audio/video device, etc.), a virtual assistant, etc. The output device 710B is configured to provide an output, as necessary, based on the estimated nervous system state provided by system 100, thereby enabling open-loop control of the nervous system state, e.g., sympathetic arousal or cortisol-related energy production, according to the estimated state provided by system 100.

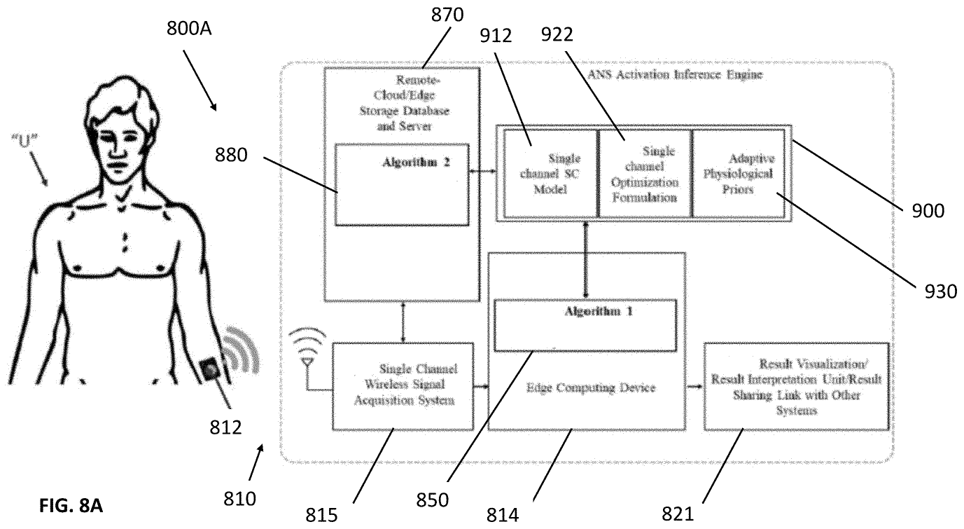

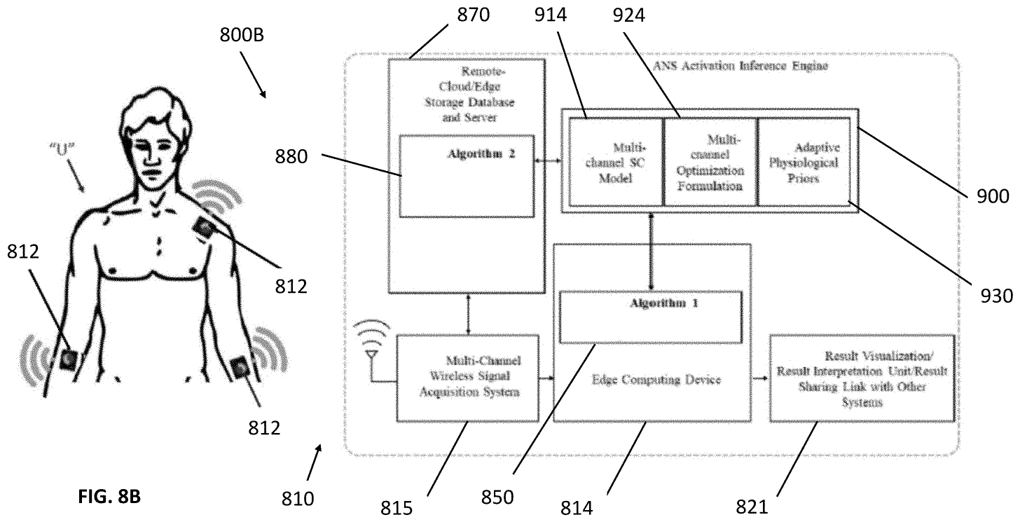

Turning to FIGS. 8A and 8B, systems 800A, 800B provided in accordance with the present disclosure are shown and described hereinbelow configured for identifying autonomic nervous system (ANS) activations based on measurement(s) of a (non-neural) physiological condition, e.g., skin conductance, for use in estimating or inferring a nervous system state such as, for example, a state of sympathetic arousal. Systems 800A, 800B are similar to one another except that system 800A (FIG. 8A) utilizes a single-channel input from a single sensor, e.g., one sensor 812 positioned on a body of a user “U,” and provides single-channel processing, while system 800B (FIG. 8B) utilizes a multi-channel input from a plurality of sensors, e.g., plural sensors 812 positioned at similar or different locations on the body of the user “U,” and multi-channel processing. Thus, except as explicitly contradicted hereinbelow, aspects and features described with respect to system 800A may similarly apply to system 800B, and vice versa. Further, systems 800A, 800B may be similar to and include any of the aspects and features of systems 100 (FIGS. 1A-2B) or 500 (FIGS. 5A and 5B) except as explicitly contradicted hereinbelow. Additionally, it is noted that the identified ANS activations, which are indicative of a nervous system state (e.g., state of sympathetic arousal), determined by systems 800A, 800B may be used as part of a feedback-based control system. Such feedback-based control systems suitable for use with systems 800A, 800B include, for example, closed-loop control system 700A (FIG. 7A), open-loop control system 700B (FIG. 7B), or any other suitable feedback-based control system.

Continuing with reference to FIGS. 8A and 8B, systems 800A, 800B each include: a sensor assembly 810 including one or more sensors 812, and a computing device 870, e.g., a smartphone, tablet, cloud server with associated storage, etc. Each sensor assembly 810 may include a single sensor 812 to obtain a single sensor reading (e.g., for single-channel input in system 800A), or may include multiple sensors 812 to enable obtaining multiple different sensor readings (e.g., for multi-channel input in system 800B). With respect to multiple sensor configurations, plural sensors 812 may be incorporated into a single device configured for positioning at one location on the user “U,” or separate devices each including one or more sensors 812 may be provided for positioning at various different locations on the user “U.” That is, sensor assembly 810 may include a single unit with one or more sensors 812 or may include a collection of operably (but not necessarily physically) coupled units each including one or more sensors 812. Sensors 812 may be any suitable skin conductance sensors, also referred to as electrodermal activity (EDA) sensors.

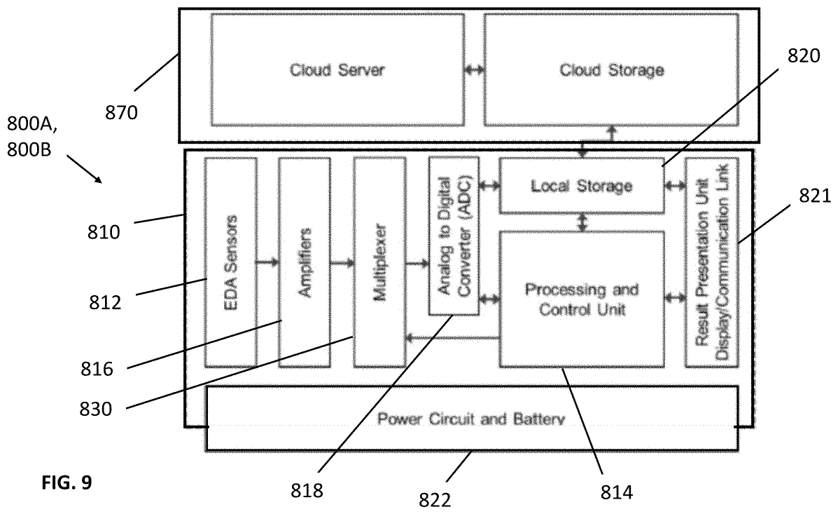

With additional reference to FIG. 9, sensor assembly 810 may be configured similar to sensor assembly 110 described above with reference to FIGS. 1A and 1B and, thus, may include one or more wearable or otherwise attachable devices positionable on the body of the user “U,” each including one or more sensors 812. Sensor assembly 810 may further include a processing and control unit 814 and intermediate circuitry 815 such as, for example, an amplifier 816, and an analog-to-digital (A/D) converter 818, and/or other suitable circuitry operably coupled between the sensor(s) 812 and a processing and control unit 814. Sensor assembly 810 may also include a local storage 820, an Input/Output (I/O) 821, and, in aspects where multiple sensors 812 are utilized (e.g., with respect to system 800B), a multiplexer 830 coupled between amplifier 816 and A/D converter 818. I/O 821 includes a user interface (UI) such as a display monitor, touch-screen display, a speaker, one or more LED's, etc., and/or is configured to communicate with a separate UI, e.g., display, smartphone or other computing device, server, printer, speaker, etc., to output information to the user “U” or other person such as a healthcare professional, family member, caregiver, etc. I/O 821 or a separate I/O is configured to facilitate communication of information between sensor assembly 810 and other local or remote devices, via wired or wireless communication.

Multiplexer 830 is configured to receive the multi-channel input, e.g., from each sensor 812 as amplified by amplifier 816, and to switch between the available amplified sensor inputs for forwarding to the A/D converter 818. Sampling by the multiplexer 830, e.g., switching between the channels, is controlled by processing and control unit 814 according to a pre-determined sampling algorithm, a dynamic sampling algorithm, or in any other suitable manner. Sensor assembly 810 also includes a power source 822, e.g., a battery, configured to power sensor assembly 810.

Sensors 812, in use, may provide voltage values that are proportional to the skin conductance values for the region of the body where each particular sensor 812 is located. Sensor 812 may also provide associated time data with the voltage values, or time data may be determined upon receipt of the voltage values at processing and control unit 814. Amplifier 816 is configured to amplify the voltage values so that they can be successfully read with A/D converter 818. If necessary, e.g., in multi-channel configurations, switching via multiplexer 822 is provided between amplification and conversion. Other suitable additional or alternative hardware processing of the raw sensor data, e.g., the voltage values, is also contemplated. The resultant amplified and converted data, referred to herein as the processed sensor data, is stored in local storage 820 (in aspects, together with time data) and may be transmitted to a remote device, e.g., directly or indirectly to computing device 870, for synchronization therewith continuously, periodically at prescribed intervals, or when the communication link between them is available. This transmission is directed by processing and control unit 814 via I/O 821, for example.

Processing and control unit 814 is configured to load the sensor data stored in local storage 820 for offline analysis and/or is configured to obtain the sensor data directly from A/D converter for real-time analysis. Processing and control unit 814 may be a microprocessor (or microcontroller unit (MCU)) with sufficient processing power for low latency real-time estimation, a dedicated System on Chip (SoC) for low-power and real-time digital signal processing (DSP), a low power digital system implementation for real-time DSP on a field programmable gate array (FPGA), an ASIC, or other suitable processing and control unit. Processing and control unit 814 may be partially or wholly integrated into one of the units of sensor assembly 810 or may be separate from some or all of the units of sensor assembly 810 and operably coupled thereto via a wired or wireless connection, directly or indirectly. Processing and control unit 814 may be embodied in a single device or incorporated across multiple devices. Processing and control unit 814 further includes a memory associated therewith which may be one or more of a volatile type memory, e.g., RAM, or a non-volatile type memory, e.g., flash media, disk media, etc. A suitable user interface as part of I/O 821 of sensor assembly 810 or separately therefrom may be provided to present status information, present results, communicate data to/from other devices, etc.

Referring still to FIGS. 8A-9, the memory of processing and control unit 814 stores a first algorithm 850 and is configured to communicate with computing device 870 which includes a memory that stores a second algorithm 880. First and second algorithms 850, 880, are configured to infer ANS activations based upon the sensor data. First algorithm 850, in aspects, may be a least squares algorithm for offline sparse recovery of ANS activation, a Bayesian filter algorithm (e.g., implemented using iteratively-reweighted Kalman filtering and backward smoothing) for offline sparse recovery of ANS activation, a Bayesian filter algorithm (e.g., implemented using iteratively-reweighted Kalman filtering and backward smoothing) for real-time sparse recovery of ANS activation, or other suitable algorithm. Second algorithm 880 may include an expectation-maximization algorithm, a coordinate-descent algorithm, or other suitable algorithm.

The first and second algorithms 850, 880 utilize a model framework 900 having a single channel skin conductance model 912 and/or a multi-channel skin conductance model 914, a single channel optimization formulation 922 and/or a multi-channel optimization formulation 924, and adaptive physiological priors 930. The first and second algorithms 850, 880 estimate model parameters, e.g., coefficients, to enable first algorithm 850 and second algorithm 880 to identify ANS activations based on the obtained sensor data. More specifically, the estimation of parameters using first algorithm 850, e.g., within sensor assembly 810 or other wearable, portable, etc. local device, may be based upon sparse recovery, which requires relatively low computational power and consumption and, thus, is suitable for being performed by sensor assembly 810, which is a relatively low performance device as necessitated by size, battery life, and other constraints. On the other hand, the estimation of parameters using second algorithm 880 may be based on expectation-maximization (EM) or coordinate-descent, which requires relatively high computational power and consumption and, thus, is more suitable for running on high performance devices such as computing device 870, e.g., a smartphone, server, tablet, etc.

Upon communication and synchronization between sensor assembly 810 and computing device 870, which may be accomplished upon connection, periodically at prescribed intervals, upon request, etc., updated parameters are communicated between first and second algorithms 850, 880, thus enabling first algorithm 850 to benefit from the relatively high computational power abilities afforded to second algorithm 880 (and the more-accurate parameters determined thereby) via occasional updating of parameters therefrom, while also being able to run effectively with relatively low computational power requirements in offline and/or real-time modes. Further, running local and remote algorithms, e.g., algorithms 850, 880, and communicating therebetween to update parameters for the local algorithm requires relatively low communication bandwidth and only occasional communication as compared to configurations wherein an algorithm(s) is only run remotely thus requiring relatively high communication bandwidth and constant or frequent communication. Communication of sensor data, results, and/or other data between sensor assembly 810 and computing device 870 may also be performed upon connection, continuously, periodically at prescribed intervals, or in any other suitable manner.

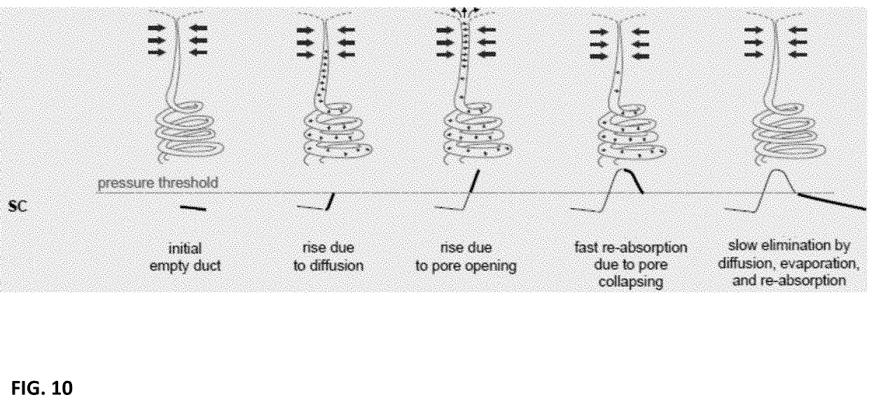

Turning to FIG. 10, the model framework 900 (FIG. 9) of the present disclosure may be based on the poral valve model, which is illustrated in FIG. 10. According to the poral valve model, an initial assumption is made that a sweat duct is initially empty and the pore is closed. At this point, the skin conductance (SC) adjacent the duct is relatively low and may be decreasing. In response to an ANS activation, secretions from the sweat gland start to fill the sweat duct. As the amount of sweat in the duct increases, there is an increase in the hydraulic pressure inside. The pressure build-up gives rise to increasing diffusion into the corneum and the deeper corneum area, resulting in a relatively slight rise in skin conductance. Once the pressure within the duct exceeds a threshold, the pore is opened to enable sweat secretion through the pore and onto the skin. The secreted sweat onto the skin contributes to a relatively sharp rise in increase in skin conductance.

The direct secretion of sweat and the diffusion of the pore reduce the hydraulic pressure previously built up and, once the pressure falls below a certain threshold, the pore collapses to a closed configuration, thereby separating the sweat contents in the duct from the skin surface and preventing the sweat contents in the duct from contributing to skin conductance. As a result, a relatively sharp decline in skin conductance is realized. This is defined as the fast reabsorption which, as noted above, due to pore collapsing, results in a fast decay of skin conductance. The remaining secreted fraction of sweat in the corneum is diffused into the deeper dermis and cleared away from the periductal area by evaporation and reabsorption. This provides a relatively slow decay of skin conductance and, thus, is defined as the slow reabsorption.

Based on the poral valve model detailed above, a nonlinear, e.g., 3D, state-space model in the form of three compartment pharmacokinetic realization of the poral valve model is developed based on Equations (27), (28), and (29) for sweat production, pore collapse, and slow reabsorption, respectively:

$$\begin{matrix}{\dot{x}}_{1} ( t ) = - \frac{1}{{τ}_{r}} {x}_{1} ( t ) + u ( t ) , & ( sweet production ) \\ {\dot{x}}_{2} ( t ) = \frac{{η}_{p} ( {x}_{1} ( t ) )}{{τ}_{r}} {x}_{1} ( t ) - \frac{1}{{τ}_{p}} {x}_{2} ( t ) , & ( pore collapse ) \\ {\dot{x}}_{3} ( t ) = \frac{{η}_{d} ( {x}_{1} ( t ) )}{{τ}_{r}} {x}_{1} ( t ) - \frac{1}{{τ}_{d}} {x}_{3} ( t ) & ( slow re \text{-} absorption )\end{matrix}$$

where x₁(t), x₂(t), and x₃(t) denote the states corresponding to the amount of sweat in the sweat ducts, in the ducts but electrically conducted to the surface due to the pore opening (contributing to the SC level), and diffused in the corneum, respectively. The states x₂(t) and x₃(t) are contributing to the rise in the SC level. τₚ denotes the fast decay time due to fast reabsorption, τ_{d} denotes the slow decay time related to slow reabsorption, and τᵣ denotes the rise time or the clearance rate of the sweat from the ducts. The system input μ(t) represents the ANS activation. ηₚ(x₁(t)) and η_{d}(x₁(t)) are functions that determine the fraction of sweat secreted by direct pore opening and diffusion, respectively.

The nonlinearities above can be modeled with sigmoid functions, followed by modeling the continuous state-space model in matrix form after several assumptions to, finally, through discretization, provide a discrete state-space model as defined in Equation (30):

xₖ=Axₖ₋₁+Buₖ,yₖ=Cxₖ+vₖ.

where xₖ, yₖ, μₖ, and vₖ denote the state vector, the observation, ANS activation, and the measurement error, respectively. The above model is exemplary, as other suitable models are also contemplated.

Appropriate physiologically motivated priors are enforced on the system unknowns, e.g., physiological parameters, during estimation to help balance between skin conductance measurement error and the model fit.

As noted above, first algorithm 850 (FIGS. 8A-8B) within sensor assembly 810 (FIGS. 8A-9) or other suitable relatively low-performance device (such as a wearable device, portable device, limited battery-powered device, etc.) may include sparse recovery and, more specifically, least-squares based sparse recovery and/or Bayesian filtering (e.g., implemented using iteratively-reweighted Kalman filtering and backward smoothing) based sparse recovery for ANS activation identification, whether in an offline mode, in the absence of parameter updating, or in a real-time mode. This sparse recovery may implement a scalable, fixed interval smoother-based sparse recovery approach and/or generalized-cross-validation may be utilized to tune the sparsity level. Sparse recovery has relatively low computational power requirements and, thus, is suitable for execution by a relatively low performance device, e.g., sensor assembly 810 (FIGS. 8A-9) or other suitable relatively low-performance device. Second algorithm 880 (FIGS. 8A-8B), on the other hand, may run both sparse recovery and physiological parameter estimation utilizing, for example, expectation-maximization (EM) based deconvolution for learning the model parameters to facilitate ANS activation identification. Other suitable estimation approaches for first algorithm 850 (FIGS. 8A-8B) and/or second algorithm 880 (FIGS. 8A-8B) are also contemplated. Further, the estimation using first algorithm 850 (FIGS. 8A-8B) and/or second algorithm 880 (FIGS. 8A-8B) may involve an initial initialization phase and a subsequent main phase for which different estimation algorithms may be utilized. Physiological parameter estimation (together with sparse recovery) has relatively high computational power requirements and, thus, is suitable for execution by a relatively high performance device, e.g., computing device 870 (FIGS. 8A-9).

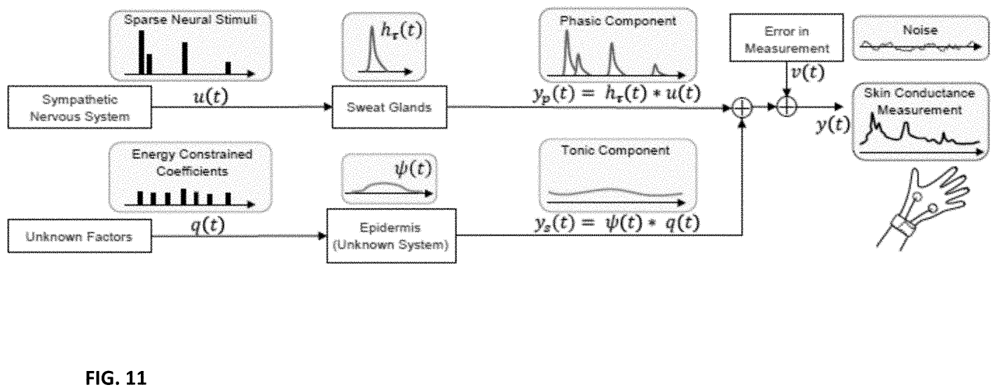

Turning to FIG. 11, in aspects, model framework 900 (FIG. 9) may be based on a sparse decomposition approach wherein a skin conductance reading (SCR) or signal is thought of as a summation of two difference components: the tonic component and the phasic component, and further includes a third component to represent noise. More specifically, as illustrated in FIG. 11, a single neural stimuli signal μ(t) generated by the sympathetic nervous system is responsible for the SCR in a particular skin region of the body. This neural stimuli μ(t) convolves with sweat glands in that particular region of the skin which has a phasic response function hᵣ(t) to generate the phasic component. The tonic component is represented as a convolution between a signal representing the weights in different time instances for the q(t) and a function ψ(t) denoting the smooth variation, while v(t) represents the measurement error. The skin conductance signal can thus be represented combining these three components in Equation (31):

y(t)=yₚ(t)+yₛ(t)+v(t)

wherein y(t), yₚ(t), yₛ(t), and v(t) represent the skin conductance signal, the phasic component, the tonic component, and noise (measurement error), respectively.

From the above, a discrete model can be deduced according to Equation (32):

$$y = \underbrace{{A}_{τ} {y}_{po} + {B}_{τ} u}_{phasic} + \underbrace{Cq}_{tonic} + ν .$$

where μ, q, and v denote the ANS activation, the coefficients of a B-spline basis function, and the noise (measurement error), respectively.

Using the above discrete model, estimation may then be performed. Estimation may involve use of a coordinate descent-based algorithm (e.g., a cross-validation-based block coordinate descent approach) implemented as algorithm 880 (FIGS. 8A and 8B) to enable simultaneously estimating the tonic component, neural stimuli, and physiological system parameters by automatically balancing the smoothness of the tonic component, the sparsity of neural stimuli, and the residual error. This has relatively high computational power requirements and, thus, is suitable for execution by a relatively high performance device, e.g., computing device 870 (FIGS. 8A-9). First algorithm 850 (FIGS. 8A-8B), similarly as above, may run sparse recovery for ANS activation identification, whether in an offline mode, in the absence of parameter updating, or in a real-time mode. This has relatively low computational power requirements and, thus, is suitable for execution by a relatively low performance device, e.g., sensor assembly 810 (FIGS. 8A-9). As also detailed above, synchronization between sensor assembly 810 and computing device 870 (see FIGS. 8A-9) enables updated parameters, e.g., the sparse coefficients, to be communicated between first and second algorithms 850, 880 (see FIGS. 8A-9) for updating first algorithm 850.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The aspects described with reference to the attached drawings are presented only to demonstrate certain examples of the disclosure. Other elements, steps, methods, and techniques that are insubstantially different from those described above and/or in the appended claims are also intended to be within the scope of the disclosure.

What is claimed is:

1. A system for estimating a state of the nervous system, comprising:
at least one sensor configured to sense a continuously variable non-neural physiological condition and to output sensed data indicative of the continuously variable non-neural physiological condition;
a relatively low performance processing device operably coupled to the at least one sensor and configured to receive the sensed data therefrom, the processing device including a processor and memory storing a first algorithm that, when executed by the processor, causes the processor to run the first algorithm to estimate a state of a nervous system based on the sensed data, the processing device configured to output the estimated state of the nervous system; and
a relatively high performance computing device operably coupled to the processing device and including a processor and memory storing a second algorithm that, when executed by the processor, causes the processor to run the second algorithm to determine updates, the computing device configured to communicate the updates to the processing device to improve the estimate of the state of the nervous system.

2. The system according to claim 1, wherein the at least one sensor includes at least one skin conductance sensor and wherein the continuously variable non-neural physiological condition is skin conductance.

3. The system according to claim 2, wherein the estimate of the state of the nervous system is at least one of: an identification of an autonomic nervous system activation or an estimate of a state of sympathetic arousal.

4. The system according to claim 1, wherein the at least one sensor includes at least one blood cortisol sensor and wherein the continuously variable non-neural physiological condition is a user's blood cortisol level.

5. The system according to claim 4, wherein the estimate of the state of the nervous system is an estimate of a state of cortisol-related energy production.

6. The system according to claim 1, wherein the processing device is further configured to receive an external input and wherein the processor is caused to estimate the state of the nervous system based on the sensed data and the external input.

7. The system according to claim 1, wherein the first algorithm is executed to estimate the state of the nervous system based on the sensed data in real-time, and wherein the second algorithm is executed to determine updates occasionally.

8. The system according to claim 1, wherein both the first and second algorithms perform estimation and, wherein, the second algorithm is utilized to provide updated parameters to the first algorithm for estimating the state of the nervous system based on the sensed data using the first algorithm.

9. The system according to claim 1, wherein the first algorithm includes a forward filter algorithm and wherein the second algorithm includes the forward filter algorithm and a backward smoothing algorithm.

10. The system according to claim 9, wherein the computing device is configured to repeatedly run the forward filter and backward smoothing algorithms to obtain updated model parameters, and wherein the updated model parameters are included in the updates provided from the computing device to the processing device.

11. The system according to claim 1, wherein the first algorithm includes a sparse recovery algorithm and wherein the second algorithm includes a sparse recovery algorithm and a further estimation algorithm.

12. The system according to claim 11, wherein the further estimation algorithm includes an expectation maximization algorithm or a coordinate descent algorithm.

13. The system according to claim 11, wherein the sparse recovery algorithm includes a least squares algorithm or a Bayesian filter algorithm.

14. The system according to claim 1, wherein at least one of the first algorithm or the second algorithm is based on a poral valve model.

15. The system according to claim 1, wherein at least one of the first algorithm or the second algorithm is based on a decomposition model wherein the sensed data is decomposed into a tonic component, a phasic component, and a noise component.

16. The system according to claim 1, wherein the first algorithm includes at least one neural network.

17. The system according to claim 16, wherein a first neural network of the at least one neural network is configured to model how the estimated nervous system state at least one of: evolves with time or relates to observations, and wherein a second neural network of the at least one neural network is configured to estimate the nervous system state.

18. The system according to claim 16, wherein the computing device is configured to re-train the at least one neural network and wherein updated neural network weights are included in the updates provided from the computing device to the processing device.

19. The system according to claim 1, further comprising:
at least one second sensor configured to sense a second continuously variable non-neural physiological condition and to output second sensed data indicative of the second continuously variable non-neural physiological condition,
wherein the first algorithm is configured to estimate the state of the nervous system based on the sensed data and the second sensed data.

20. The system according to claim 19, wherein the first and second continuously variable non-neural physiological conditions are skin conductance at different bodily locations.

21. The system according to claim 19, wherein the first continuously variable non-neural physiological condition is skin conductance and wherein the second continuously variable non-neural physiological condition is heart rate.

22. A control system, comprising:
the system according to claim 1; and at least one of:
a therapy-providing device configured to receive the estimated state of the nervous system output from the processing device and to provide a therapy to a user based thereon; or
an output device configured to receive the estimated state of the nervous system output from the processing device and to provide an output based thereon.

23. A method for estimating a state of the nervous system, comprising:
obtaining sensed data indicative of a continuously variable non-neural physiological condition;
estimating, using a relatively low performance device, a state of a nervous system based on the sensed data;
outputting the estimated state of the nervous system; and
receiving, at the relatively low performance device, updates from a relatively high performance device to improve the estimating.

24. The method according to claim 23, wherein obtaining the sensed data includes obtaining skin conductance data from at least one skin conductance sensor, and wherein estimating the state of the nervous system includes: an identification of an autonomic nervous system activation estimating a state of sympathetic arousal.

25. The method according to claim 23, wherein obtaining the sensed data includes obtaining blood cortisol level data, and wherein estimating the state of the nervous system includes estimating a state of cortisol-related energy production.

26. The method according to claim 23, further comprising: receiving an external input, and wherein the estimating is based on the sensed data and the external input.

27. The method according to claim 23, further comprising: estimating, at the relatively high performance device, the state of the nervous system based on the sensed data and determining updated parameters based upon the estimating, wherein the updates include the updated parameters.

28. The method according to claim 23, wherein the estimating is performed in real-time and wherein the updates are received occasionally.

29. The method according to claim 23, wherein the estimating includes a sparse recovery algorithm and wherein the updates are based at least partially on a sparse recovery algorithm and one of: an expectation maximization algorithm or a coordinate descent algorithm.

30. The method according to claim 29, wherein the sparse recovery algorithm includes a least squares algorithm or a Bayesian filter algorithm.

31. The method according to claim 23, wherein at least one of the estimating or the updates are based on a poral valve model.

32. The method according to claim 23, wherein at least one of the estimating or the updates are based on a decomposition model wherein the sensed data is decomposed into a tonic component, a phasic component, and a noise component.

33. The method according to claim 23, wherein the estimating includes running a forward filter algorithm, and wherein the updates include updated model parameters determined by repeatedly running the forward filter algorithm and a backward smoothing algorithm for the forward filter algorithm.

34. The method according to claim 23, wherein the updates include updated model parameters determined by repeatedly running an expectation-maximization algorithm.

35. The method according to claim 23, wherein the estimating includes running a first neural network to model how the estimated nervous system state at least one of: evolves with time or relates to observations, and running a second neural network to estimate the nervous system state.

36. The method according to claim 35, wherein the updates include updated neural network weights.

37. The method according to claim 36, wherein the updated neural network weights are determined by re-training the at least one neural network.

38. The method according to claim 23, further comprising:
obtaining second sensed data indicative of a second continuously variable non-neural physiological condition, and wherein the estimating is based on the sensed data and the second sensed data.

39. The method according to claim 38, wherein the first and second continuously variable non-neural physiological conditions are skin conductance at different bodily locations.

40. A method of control, comprising:
the method according to claim 23;
receiving the estimated state of the nervous system; and at least one of:
providing a therapy to a user based on the received estimated state of the nervous system; or
providing an output based on the received estimated state of the nervous system.
